(19) United States
(12) Patent Application Publication
(10) Pub. No.: US 2026/0321437 A1
(43) Pub. Date: Oct. 15, 2026

(54) DIGITAL SYSTEMS AND METHODS FOR NAVIGATING AND MANAGING CLINICAL CARE CONTINUUM

(71) Applicant: MANTA CARES INC., San Francisco, CA (US)

(72) Inventors: Samira Sunil DASWANI, San Francisco, CA (US); Douglas BLAYNEY, San Francisco, CA (US)

(21) Appl. No.: 19/481,086

(22) PCT Filed: May 3, 2024
(86) PCT No.: PCT/US2024/027799

§ 371 Date: Nov. 3, 2025

Related U.S. Application Data
(60) Provisional application No. 63/500,852, filed on May 8, 2023.

(51) Int. Cl. G16H 50/70; G16H 70/20

(52) U.S. Cl. CPC G16H 50/70; G16H 70/20

(57) ABSTRACT

The subject technology is directed to systems and methods for navigating and managing clinical care continuum related to a digital platform that enables patients and healthcare professionals to navigate complex treatment journeys with ease. The system includes a pathway modeling system that generates a visual representation of the treatment landscape and potential journeys as a pathway map within a user interface, complete with a set of “stations” that correspond to different phases in the journey.

### CROSS-REFERENCE TO RELATED APPLICATIONS

This application claims priority to U.S. Provisional Application No. 63/500,825, filed May 8, 2023, which is commonly owned and incorporated by reference herein for all purposes.

### TECHNICAL FIELD

This invention relates generally to the field of digital medical systems and more specifically to a new and useful digital system and method for navigating and managing clinical care continuum.

### BACKGROUND OF THE INVENTION

In the modern healthcare landscape, patients are often faced with complex and difficult treatment decisions. These decisions can be particularly overwhelming in cases such as cancer care, where patients and their caregivers may be faced with a multitude of treatment options, each with its own set of risks and benefits. Additionally, the medical knowledge base is rapidly expanding (e.g., doubling approximately every 73 days), which poses a significant challenge not only to patients but also to healthcare providers. This rapid growth in knowledge makes it increasingly difficult for healthcare professionals to keep up to date, thereby complicating their ability to access the right information when needed to make informed treatment decisions for their patients.

Navigating this landscape of treatment options and decisions requires a significant amount of time and effort from both patients and healthcare providers. Patients often must rely on various information sources to understand their options, including medical professionals, academic articles, and online resources, which can be difficult to understand and interpret, and can lead to uninformed or misinformed decisions. As a result, there is a need for an easier and more efficient way for patients and healthcare providers to understand the treatment options, communicate with one another, and navigate through the complexities of various treatment journeys.

Thus, there is a need in the digital medical system field to create a new and useful system and method for navigating and managing clinical care continuum. This invention provides such a new and useful system and method.

### BRIEF DESCRIPTION OF DRAWINGS

FIG. 1 is a schematic representation illustrating the structure of a pathway map down to an individual stage and ways of dynamically personalizing a path according to embodiments of the subject technology.

FIG. 2 is an exemplary representation of a portion of a pathway map within one zone according to embodiments of the subject technology.

FIGS. 3A-3B are simplified flow diagrams illustrating a method for navigating and managing clinical care continuum according to embodiments of the subject technology.

FIG. 4 is a flowchart representation of a method for navigating and managing clinical care continuum according to embodiments of the subject technology.

FIG. 5 is a chart outlining one exemplary implementation of a pathway map and associated modules with it according to embodiments of the subject technology.

FIG. 6 is a schematic representation of a system for navigating and managing clinical care continuum according to embodiments of the subject technology.

FIG. 7 is an exemplary system architecture that may be used in implementing the system and/or method according to embodiments of the subject technology.

FIG. 8 is an exemplary pathway map segment illustrating a diagnosis phase for early-stage estrogen receptor (ER)/progesterone receptor (PR) positive (ER+/PR+) breast cancer according to embodiments of the subject technology.

### DETAILED DESCRIPTION OF THE EMBODIMENTS

The following description of the embodiments of the invention is not intended to limit the invention to these embodiments but rather to enable a person skilled in the art to make and use this invention.

One general aspect includes an apparatus for navigating and managing clinical care continuum. For instance, the term “clinical care continuum” may refer to the entire spectrum of clinical experiences, which may include, without limitation, preventive care, diagnosis, treatment, survivorship, end-of-life care, and/or the like. The apparatus may include a storage medium configured to store a first database, the first database comprising a first data associated with first clinical information. The term “clinical information” may refer to various data related to patient health, medical interventions, and/or healthcare processes. Clinical information may include, without limitation, treatment guidelines, clinical practice, patient experience data, diagnostic records, health metrics, research findings, and/or the like.

The apparatus further may include a processing unit coupled to the storage medium. The processing unit may be configured to retrieve the first data from the storage medium and generate a pathway map using at least the first data. The pathway map may represent a hierarchy of clinical stages within a first clinical process. For example, the term “clinical stage” may refer to specific phases or points in time within the clinical care continuum, which may include, without limitation, symptom screening, diagnostic testing, diagnosis confirmation, treatment planning, monitoring, and/or the like. The term “clinical process” may refer to a sequence of clinical care management, which may include one or more clinical stages. Clinical process may include, without limitation, preventive care, diagnosis, treatment, survivorship, end-of-life care, and/or the like.

In some embodiments, the apparatus may further include a memory coupled to the processing unit and configured to store the pathway map. The apparatus may further include a user interface coupled to the processing unit and configured to receive a first user input. The apparatus may further include a network device coupled to the processing unit and configured to communicate with a second database.

Implementations may include one or more of the following features. The pathway map may comprise a first stage of the first process, and the first stage comprises a first module configured to provide medical information associated with the first stage and a medical condition for a first patient. The medical information may comprise at least one of treatment options, educational resources, risk assessments, nutritional guidance, support services, or medication information. The processing unit may be further configured to update the pathway map based on a first parameter, the first parameter being associated with the first user input. The first user input may comprise at least one of stage identification, medical diagnosis, diagnostic test result, treatment decision, symptom report, or user preference. The processing unit may be further configured to update the pathway map based on a second parameter associated with an update of the first data. In some embodiments, the second database comprises a second data associated with patient-specific information, the patient-specific information comprises at least one of medical history, genetic information, biomarker information, diagnostic results, current medications, allergy information, financial information, or insurance information. The user interface may further comprise a display device configured to display the pathway map.

According to another embodiment, the subject technology provides a system for navigating and managing clinical care continuum. The system may include a pathway modeling subsystem configured to generate a pathway map representing a hierarchy of clinical stages within a first clinical process, the pathway map comprising a first stage indicative of a first step of the first clinical process. The system may further include a user interaction subsystem configured to display the pathway map and receive a first user input. The system may further include a module customization subsystem configured to update the first stage based on the first user input. The system may further include a communication subsystem configured to communicate with an external database.

Implementations may include one or more of the following features. The first stage may comprise a decision station or a scenario station. The first stage may comprise a first module configured to provide medical information associated with the first stage. The medical information may comprise at least one of treatment options, educational resources, risk assessments, nutritional guidance, support services, or medication information. The system may further comprise an account management subsystem configured to manage user profiles and permissions for accessing the pathway map. The system may further comprise a data integration subsystem configured to integrate with the external database, the external database comprises at least one of electronic health records, payor portals, or prescription systems. The pathway map may be generated based on first clinical information. The system may further comprise an information processing engine configured to perform content adaptation within the pathway map based on user preference.

According to yet another embodiment, the subject technology provides a method for navigating and managing clinical care continuum. The method may include generating, by a processing unit, a pathway map representing a hierarchy of clinical stages within a first clinical process, the pathway map comprising a first stage indicative of a first step of the first clinical process. The method may further include displaying, by a user interface, the pathway map. The method may further include receiving, by the user interface, a first user input. The method may further include updating, by the processing unit, the pathway map based on a first parameter associated with the first user input.

Implementations may include one or more of the following features. The method may further include providing, by the user interface, medical information associated with the first stage. The medical information may comprise at least one of treatment options, educational resources, risk assessments, nutritional guidance, support services, or medication information. The first user input comprises at least one of stage identification, medical diagnosis, diagnostic test result, treatment decision, symptom report, or user preference.

According to further embodiments, the system for navigating and managing the clinical care continuum may be configured to identify a current location within the pathway map based on a user input. The system may dynamically reveal possible paths on the pathway map based on the current location. In some examples, the system may be configured to receive a search query and highlight one or more stations associated with the search query on the pathway map. The system may also include a chatbot feature configured to provide interactive support to the user. In some cases, each station on the pathway map may display the estimated time and duration associated with each stage of the clinical process. These estimates may be calculated based on the specific treatments or procedures planned at each stage and can dynamically adjust in response to user inputs or updates to treatment guidelines. The system may also calculate and display associated dates for each stage, helping users anticipate future stages and associated requirements.

### 1. Overview

Systems and methods for navigating and managing clinical care continuum relates to a digital platform that enables users (e.g., patients, healthcare professionals, caregivers, third-party companies, insurance providers, pharmaceutical companies, research institutions, policymakers, and/or the like) to navigate complex treatment journeys with ease. The system includes a pathway modeling system that generates a visual representation of the treatment landscape and potential journeys as a pathway map within a user interface, complete with a set of “stations” that correspond to different phases in the journey.

At each station, the platform provides users with access to a range of modules that can be used to make informed decisions, take specific actions related to the treatment and access relevant resources to help manage the journey. Importantly, the modules available at each station may be customizable to address the needs of patients with different healthcare conditions, resulting in a highly tailored and personalized experience.

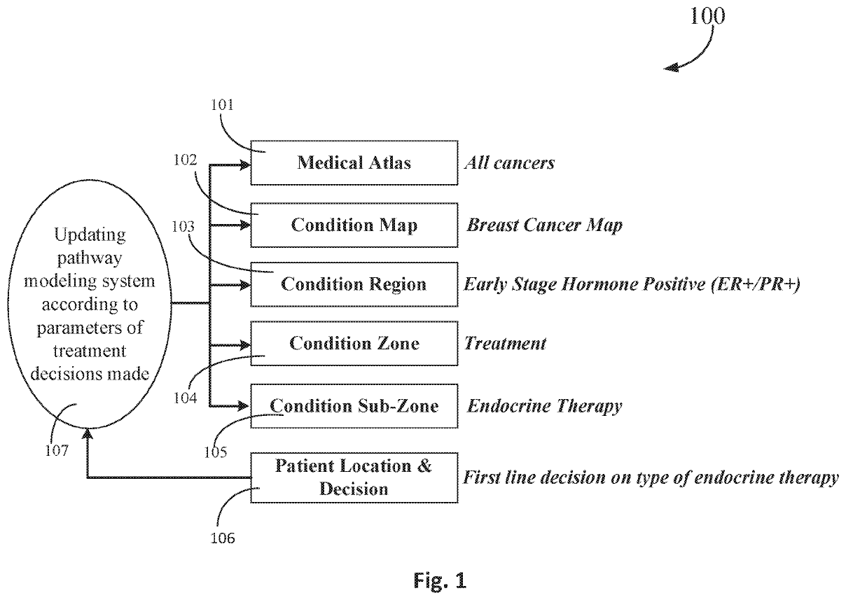

FIG. 1 is a schematic representation illustrating structure of pathway map 100 down to an individual stage and ways of dynamically personalizing a path according to embodiments of the subject technology. This diagram is merely an example, which should not unduly limit the scope of the claims. One of ordinary skill in the art would recognize many variations, alternatives, and modifications. The pathway map 100 may be adapted for various medical conditions and phases of treatment. It allows for seamless navigation across different clinical stages and provides clarity at each decision point.

Pathway map 100 and the set of different stations enable a user to better understand where they are in the clinical process, make better-informed decisions about treatment options, track symptoms and other aspects related to their illness or treatment, and access resources. In various implementations, pathway map 100 may be used to organize a clinical process into several hierarchical levels, from a broad overview down to specific patient decisions. As shown in FIG. 1, the systems and methods may provide dynamic context to a user at different levels of abstraction. For example, pathway map 100 includes medical atlas 101, which provides a comprehensive directory of all known cancers. Medical atlas 101 may act as an entry point, guiding users from the broadest category of cancers to specific condition map 102 based on the user's specific medical conditions. As an example, a user with a focus on breast cancer may be guided to a breast cancer map, which provides a detailed overview of breast cancer including, without limitation, breast cancer subtypes, diagnostic approaches, treatment protocols, statistical outcomes, and/or the like.

Depending on the specificity of the user's medical condition, condition region 103 further narrows down the pathway by focusing on a specific area of condition map 102. For instance, a user identified with early-stage hormone-positive breast cancer (ER+/PR+) may navigate to a condition region specifically curated for this subtype, allowing for a more concentrated and relevant set of data and resources. In some examples, condition zone 104 further divides the patient journey into one or more segments such as diagnosis, active treatment, survivorship, and/or the like. A diagnosis zone may provide information on screening tests and interpretation of results. A treatment zone may offer insights into different therapeutic approaches, side-effect management, and monitoring protocols. A survivorship zone may focus on follow-up care, lifestyle adaptations, and long-term health maintenance.

In various implementations, condition zone 104 may include one or more condition sub-zones 105, depending on the complexity and breadth of treatment options within the particular zone. For instance, within a treatment zone dedicated to managing and evaluating treatment options, an endocrine therapy sub-zone may be provided for patients undergoing hormone therapy as part of their breast cancer treatment. The endocrine therapy sub-zone may provide detailed information regarding different hormone therapies, potential side effects, selection of medications, and/or efficacy rates. The user may leverage this detailed information to make informed treatment decisions and identify their precise location 106 within pathway map 100 (e.g., the specific stage during the clinical process). These subzones allow users to delve deeper into a selected area of the map, providing detailed sub-maps that offer comprehensive information tailored to their specific needs only when necessary. This not only enhances the user experience by minimizing information overload but also ensures that each user receives the information most relevant to their stage of clinical care, facilitating more informed decision-making.

In certain embodiments, users may customize pathway map 100 based on individual health factors and treatment responses. For instance, a user may mark their selected endocrine therapy choice on the map through user interface module 107, and the pathway modeling system may update pathway map 100 according to the parameters of treatment decisions made, thereby reflecting a personalized treatment pathway that aligns with the patient's unique health profile and preferences. Pathway map 100 allows for a personalized navigation experience, as users can visualize and understand the treatment pathway tailored to their unique medical condition, equipping them with the knowledge to discuss their care plan with healthcare providers effectively. The systems and methods can enable more efficient coordination between patients, doctors, nurses, other members of the medical team, third party payers, and caregivers.

According to some embodiments, the systems and methods may convert treatment guidelines into algorithmic and navigational logic. The systems and methods may be configured or modified for use with a variety of different clinical treatments. For example, different varieties of cancer could each have their own clinical treatment templates such that cancer patients can have highly specialized pathway maps to help navigate their treatment journey.

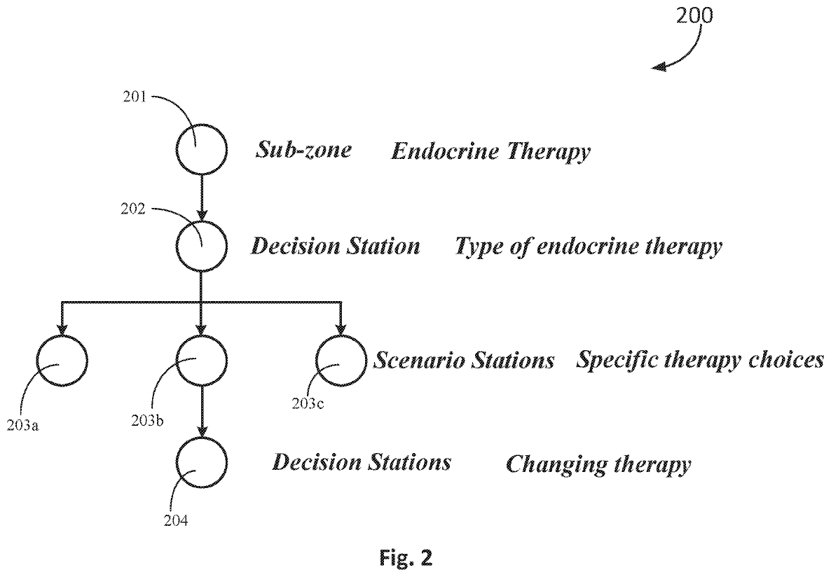

FIG. 2 is an exemplary representation of a portion of pathway map 200 within one zone according to embodiments of the subject technology. This diagram is merely an example, which should not unduly limit the scope of the claims. One of ordinary skill in the art would recognize many variations, alternatives, and modifications. Pathway map 200 may include one or more sub-zones 201, depending on the complexity and breadth of treatment options. Sub-zone 201 may provide focused information and guidance on particular aspects of treatment, such as potential therapeutic approaches, medication profiles, medical considerations, and/or the like.

As shown in FIG. 2, moving through these different stations (or stages), a patient needing endocrine therapy may be supported by one decision-focused station 202 that helps in deciding a type of endocrine therapy, a set of different scenario stations 203a, 203b, 203c specific to different therapy choices, and possibly another decision station 204 to help in deciding on changing therapy.

The systems and methods can include account management systems which may be used to manage how different entities may access the digital platform. The systems and methods may support user interfaces and functionality directed at patients. For instance, a patient-directed interface may include visual indicators of progress, interactive elements for inputting user data, and personalized notifications to guide the patient through their treatment journey. The systems and methods may additionally include user interfaces and functionality directed at doctors, caregivers, family members and others with an interest in the individual care of a patient. For healthcare professionals, the user interface may allow for the integration of electronic medical records, providing a comprehensive view of the patient's medical history, current conditions, and ongoing treatments. In some cases, the user interface may include communication modules for secure messaging, consultation scheduling, and remote patient monitoring, thereby increasing the efficiency of healthcare delivery and patient engagement. The system may also generate relevant documentation for healthcare providers to submit to insurance groups to claim reimbursement for patient services, including, but not limited to, patient illness navigation codes, treatment logs, procedure verification forms, medication administration records, and/or the like. Additionally, caregivers and family members may be granted access through user-specific portals that support them in their caregiving roles, offering necessary resources and communication tools to participate effectively in the patient's clinical process.

Some variations of the systems and methods may additionally support accounts for other types of entities. These “third-party” account types may be used for insurance companies, pharmaceutical companies, hospital organizations, and/or other entities. They may use the digital platform to gain insight into more group-level data of treatment paths. They may also use the digital platform to set or configure different modules to provide enhanced functionality related to their particular interests. For example, an insurance module may be configured to provide insurance related information to help people evaluate how their insurance coverage alters different treatment options. In another example, a pharmaceutical company may provide customized modules to better inform doctors and patients as to pharmaceutical options during their treatment.

The system and method may provide a number of potential benefits. The system and method are not limited to always providing such benefits and are presented only as exemplary representations for how the system and method may be put to use. The list of benefits is not intended to be exhaustive and other benefits may additionally or alternatively exist.

As one potential, the system and method may provide detailed and customized support across the treatment journey. The system and method can facilitate dynamically updating digital resources and requests for input on different decisions. This can provide appropriate prompts based on the particular situation of each patient. Additionally, the medical information provided within the system may be regularly reviewed and updated by healthcare providers to ensure accuracy and relevance. Furthermore, the support services may be enhanced by a recommendation engine, which incorporates community input to help identify additional resources that can assist the patient throughout the clinical process.

As another potential benefit, the system and method may offer significant potential benefits for patients facing complex and challenging treatment journeys. For example, cancer treatments and inflammatory bowel disease can involve long and complex treatment regimens, with many different decisions to be made along the way. With the platform's customization options for dynamically updating digital resources and requests for input on different decisions, patients can receive appropriate prompts based on their particular situation and make informed decisions at each step of the treatment journey. This level of guidance and support can help to reduce stress and anxiety for patients and caregivers alike and ultimately lead to better treatment outcomes for a wide variety of clinical treatments.

As another potential benefit, the system and method may be flexible and adaptable. The platform can be customized for different uses, depending on the needs of patients and healthcare providers. For example, the platform could enable insurance-based customizations, helping caregivers and patients make decisions based on options that are tailored to their specific insurance offerings. Additionally, the platform can be adapted to meet the specific needs of different stakeholders, such as medical professionals, patients, and caregivers.

### 2. Method

In operation, the method enables a digital platform that facilitates processes that functions to facilitate a digital experience that involves: generating a visual representation of a treatment journey (i.e., a clinical treatment pathway) with a plurality of stations (i.e., stage), each station indicative of a step in the clinical process; and dynamically providing a set of modules to the patient at each station based on information regarding the patient's condition, with each module customized to help the patient take actions or make informed decisions related to their specific treatment journey or access relevant resources to help them manage the condition.

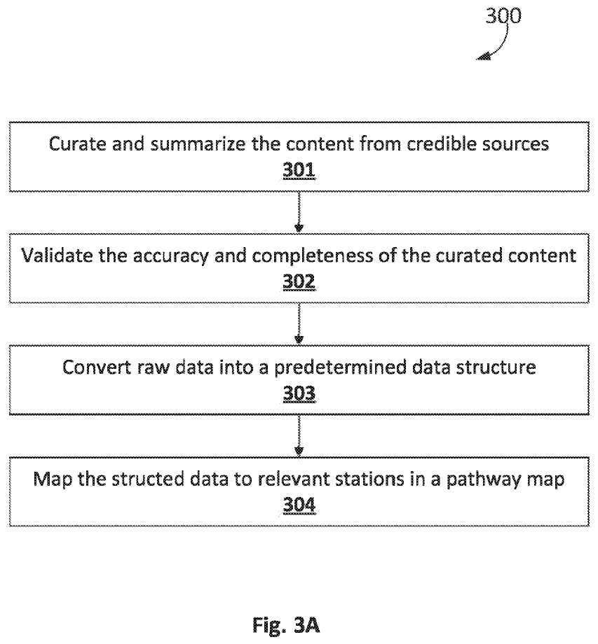

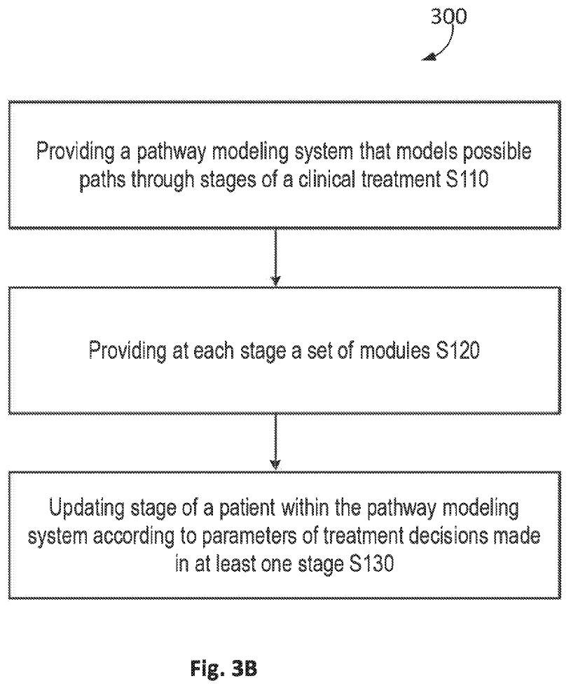

FIGS. 3A-3B are simplified flow diagrams illustrating method 300 for navigating and managing clinical care continuum according to embodiments of the subject technology. These diagrams are merely examples, which should not unduly limit the scope of the claims. One of ordinary skill in the art would recognize many variations, alternatives, and modifications. For example, one or more steps may be added, removed, repeated, modified, replaced, overlapped, and/or rearranged, and should not limit the scope of the claims. The methods and processes described herein are integrally tied to a computing system comprising one or more computing devices. Such methods may be operationalized through various forms of hardware and software implementations, such as computer application programs, application programming interfaces (APIs), libraries, and/or other computer-program product formats designed to carry out the tasks associated with managing clinical care continuum.

In various implementations, method 300 includes generating a pathway map. For example, the pathway map may represent a hierarchy of clinical stages within a first clinical process, providing a structured visualization of the treatment journey. As shown in FIG. 3A, the generation of a pathway map may include step 301 of curating and summarizing the content from credible sources including, without limitation, patient medical records, treatment guidelines, standard treatment protocols, clinical expertise, and/or the like. This step involves a comprehensive review and consolidation of current medical knowledge, guidelines, and research to compile trustworthy and actionable information for patient guidance and healthcare decision-making. Moreover, the pathway map may be developed in collaboration with clinicians to ensure that it accurately reflects real-world clinical practice patterns, thereby enhancing the relevance and applicability of the pathway maps for healthcare professionals.

In various embodiments, the pathway map may be based on a broad body of guidelines that cover various medical conditions including, but not limited to early-stage hormone-positive breast cancer, metastatic lung cancer, other subtypes or varieties of cancer, autoimmune conditions (e.g., lupus), rheumatoid arthritis, type 1 or type 2 diabetes, cardiovascular diseases, inflammatory bowel disease (IBD), chronic respiratory diseases, mental health disorders, and/or the like. In an example, the pathway map may be generated based on a treatment guideline, such as the American Society of Clinical Oncology (ASCO) guidelines. Depending on the application, the content may be summarized to a level that is comprehensible to a general audience (e.g., at a 7^{th} grade reading level) using various techniques such as natural language processing (NLP), machine learning (ML) algorithms, and/or the like.

In some embodiments, the system may be customized to tailor pathway maps for specific hospital systems or to adapt specific treatments for pharmaceutical companies. For instance, healthcare administrators or clinical teams can import hospital-specific guidelines into the system, which are then mapped to the corresponding stages within the pathway map. For pharmaceutical companies, the system may offer options to incorporate drug-specific information, such as dosing schedules, side effects, and regulatory compliance details, based on the latest clinical trials and approvals. This ensures that the pathway map remains up to date with the latest medical practices, thereby enhancing its utility across various clinical settings.

In step 302, method 300 further includes validating the accuracy and completeness of the curated content. This may involve cross-referencing the structured data against the source material to ensure fidelity to the original content. Automated algorithms may be employed to detect discrepancies or inconsistencies within the data. In some examples, the validation process may include checks by medical professionals to affirm that the synthesized data meets clinical standards and reflects current medical knowledge and practice guidelines.

In step 303, the raw data may be converted to a predetermined data structure format suitable for subsequent processing. For instance, the raw data may be converted from a comma-separated values (CSV) format to a YAML data structure, which allows for enhanced data serialization and easy readability for both machines and humans.

In step 304, the structured data may be mapped to relevant stations—which represent key decision points or treatment steps—within a pathway map. This may be achieved through a combination of algorithmic processing and predefined mapping rules that assign specific pieces of information to corresponding locations within the pathway map. For instance, information on treatment options and diagnostic tests may be mapped to the early stages of the pathway map, and follow-up care and survivorship resources may be placed in later stages.

As shown in FIG. 3B, a method for a digital platform assisting in managing clinical care continuum may include providing a pathway modeling system that models possible paths through stages of a clinical treatment S110; providing at each stage a set of modules S120; and updating stage of a patient within the pathway modeling system according to parameters of treatment decisions made in at least one stage S130. Depending on the implementation, one or more stages of the pathway map may be updated or modified based on various parameters, which may include, without limitation, direct user input (e.g., patient-reported outcomes, physician assessments, lab test results, or other clinical data entries) or external database updates (e.g., treatment guideline updates, health records updates, research findings, clinical practice, regulatory approvals), and/or the like.

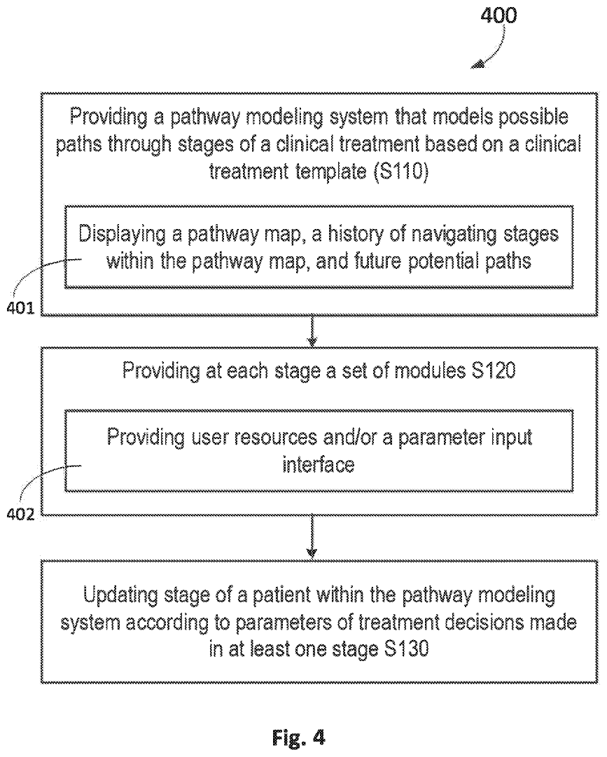

FIG. 4 is a flowchart representation of method 400 for navigating and managing clinical care continuum according to embodiments of the subject technology. This diagram is merely an example, which should not unduly limit the scope of the claims. One of ordinary skill in the art would recognize many variations, alternatives, and modifications. For example, one or more steps may be added, removed, repeated, modified, replaced, overlapped, and/or rearranged, and should not limit the scope of the claims.

The method preferably enables a user to have some representation of where they are in the broader treatment journey, but also has highly granular and specific resources for each stage of treatment. Accordingly, as shown in FIG. 4, method 400 may include: providing a pathway modeling system that models possible paths through stages of a clinical treatment based on accepted clinical guidelines S110, which comprises step 401 of displaying a pathway map and a history of navigating stages within the pathway map. For instance, the pathway map may include a first stage indicative of a first step of a clinical process.

In some embodiments, method 400 may further include providing at each stage a set of modules S120, which may include step 402, for at least one module: providing user resources and/or a parameter input interface. At each stage of the pathway map, users can access a set of modules designed to provide various resources relevant to the specific stage. These resources may include, without limitation, educational content, decision aids, symptom-tracking tools, links to external support services, and/or the like. In some examples, the first stage includes a first module configured to provide medical information associated with the first stage. This ensures that the right medical information is provided at the right time to the right patient, thus optimizing treatment outcomes and enhancing patient care. The medical information includes at least one of treatment options, educational resources, risk assessments, nutritional guidance, support services, or medication information. Depending on the implementation, the medical information may be curated from reliable sources or created by the pathway modeling system. In various examples, method 400 may further include updating stage of a patient within the pathway modeling system according to parameters of treatment decisions made in at least one stage S130.

In some variations, method 400 may additionally include providing an account management system for the pathway modeling system and wherein displaying the pathway map and a history of navigating stages within the pathway map is based at least in part on account type of a user accessing the pathway modeling system. These variations may involve exposing different views and/or different actions.

The account management system may include account types customized for patients (e.g., receivers of the treatment), doctors, and/or caregivers (e.g., family members), and these different account types may be provided with differing views and possible capabilities.

Some method variations may also include third-party accounts for entities such as insurance companies, pharmaceutical companies, and the like, and they may similarly have different access permissions. For example, such third-party accounts may have anonymized access or group-data views into the treatment pathways of a plurality of patients.

Block S110, which includes providing a pathway modeling system that models possible paths through stages of clinical treatment, functions to operate a digital platform that manages the operation of a user interface with an interactive pathway map. In some examples, the pathway map may facilitate communication between patients and healthcare providers. It may act as a visual aid during consultations, allowing healthcare providers to graphically explain the course of treatment, such as potential outcomes and necessary medical interventions, making it easier for patients to grasp their treatment journey.

Providing the pathway modeling system may include displaying a pathway map. The pathway map serves as a graphical representation of clinical treatment possibilities. The pathway map is preferably generalized and organized into different stages. The visual representation of the pathway map in one implementation may be analogous to a subway map with different paths to as a patient progresses through treatment.

In some implementations, the pathway map may be displayed to users via a user interface. Displaying the pathway map preferably also shows the path of a patient through the pathway map. A user interface may represent different stages with graphical indication of the stages the patient has previously passed, the current stage where the patient is at, and possible future stages. Displaying the pathway map not only provides a visual representation of the treatment journey but also facilitates active user interaction and customization, enhancing the utility of the map as a personalized planning tool. Through the user interface, patients can actively engage with the map, marking their current position in the treatment journey and planning for upcoming stages. This interactive feature allows patients to visually track their progress, from stages they have successfully navigated through to their present position, and anticipate future steps in their clinical process.

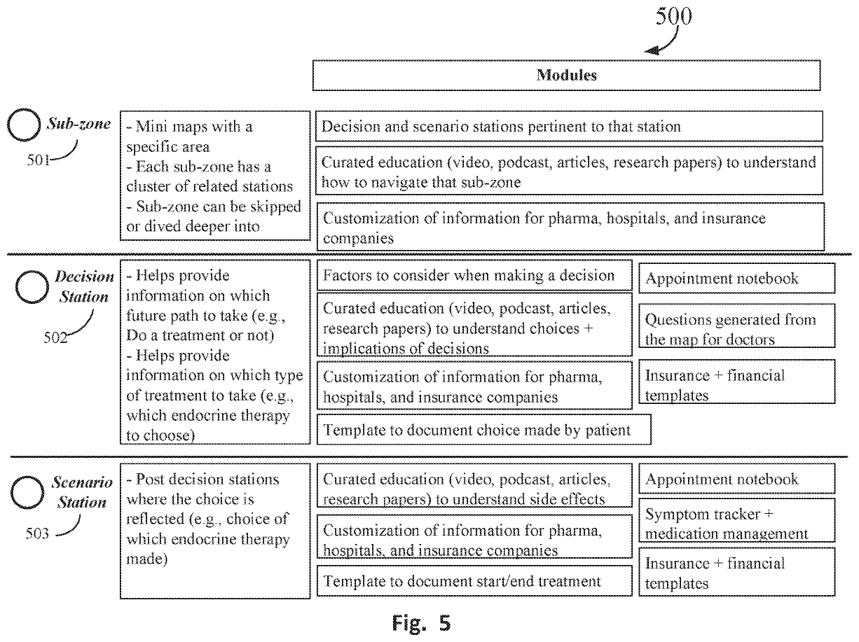

FIG. 5 is a chart outlining one exemplary implementation of pathway map 500 and associated modules with it according to embodiments of the subject technology. This diagram is merely an example, which should not unduly limit the scope of the claims. One of ordinary skill in the art would recognize many variations, alternatives, and modifications. Pathway map 500 may include one or more sub-zones 501, depending on the complexity and breadth of treatment options. Sub-zone 501 may include one or more stages (or “stations”) to provide targeted information, resources, and tools for treatment planning and management. In one variation, the pathway map can include two varieties of stages (or “stations”): a decision station 502 and a scenario station 503, and these may be organized within regions or zones as shown in FIG. 5.

A decision stage 502 functions to provide information on which future path to take (e.g., selecting a treatment). For example, this may be used to help a patient decide which endocrine therapy to choose. It may present factors to consider, include curated education for understanding choices and the impact of choices. In some variations, a dynamic information processing engine (e.g., ML/AI models) may be used to dynamically modify the information for the intended user. This may be used for dynamic summarization, explanation, or altering content for different technical levels. The decision state can also include a template of input fields to document choices.

Some modules for a decision stage may include or provide features like an appointment notebook, generated questions for an appointment, insurance/financial tools, and the like.

A scenario stage 503 may be used to reflect where a treatment choice is reflected. The scenario stage functions to provide resources and tools while a patient is going through a selected path. It could similarly provide curated educational resources. It may also include input options to document the properties of the selected treatment. It could similarly have dynamic modules providing tools like an appointment notebook, symptom tracking, medication management tracking, insurance/financial tools, and the like.

In some variations, the pathway map may include regions or zones. The regions, zones, or other types of sub-regions may function as groupings of related stages. The user interface may update to reflect a patient moving through these different regions. For example, a clinical treatment may include regions for diagnosis, treatment, and/or survivorship. In another example, at the end of acute care for a cancer, you transition to survivorship where you are doing more screening and surveillance. In a car-crash example, the person can go from surgery into long-term care, where it's about rehab and physical therapy.

Within each sub-region, there may be various other stages that a patient moves through. As shown in FIG. 2, a patient needing endocrine therapy may progress through various stages, some customized for decisions and some for support during different scenarios.

The pathway map may be interactive. A user may be enabled to adjust the scale of the pathway map. As shown in FIG. 1, there may be different levels of abstraction for the pathway. In one variation, the labels for the stages, pathways, and other options may dynamically adjust according to a zoom scale of the pathway map. For example, when zoomed out, the pathway map may be simplified to show the high-level paths—parallel paths may be consolidated into a generalized path, labels may be generalized and consolidated. In this example, when zoomed in, more detailed pathway detail with more granular stages may be presented—labels may become more detailed and broken down into more granular detail. Generative language models or other natural language modeling systems may be used to facilitate dynamic modification. For example, a clinical treatment template may provide a granular base level of detail, and a dynamic language model may be used to generate summarization and dynamic grouping of concepts when zooming out.

The user interface for the pathway map may also include other interactive features that can help patients and healthcare providers navigate and understand complex treatment options. For instance, the platform may enable users to filter the pathway map to show only the stages or modules that are relevant to their specific situation, allowing them to focus on the most critical elements of their treatment journey. Additionally, the interface may provide access to additional resources, such as educational materials and informative videos, to help patients and caregivers better understand their condition and treatment options. In some implementations, the user interface may include an AI-driven chatbot feature designed to assist users in navigating the pathway map. A chatbot may interact with users in a conversational manner, offering personalized guidance, answering questions, providing explanations for various stages of the clinical process, and suggesting the next steps based on the current progress within the pathway map.

A pathway map is preferably based on a clinical treatment template. The clinical treatment template may be configured to define the generic pathway map for a particular type or class of clinical treatments. In implementation, the clinical treatment templates may be data configuration files that incorporate expert opinion on the standard of care.

They may reflect treatment guidelines (e.g., ASCO guidelines). There may be a unique clinical treatment template for each supported clinical treatment. In one variation, the method enables patients suffering from a variety of issues make use of the digital platform for their treatment. The types of clinical treatments that may be supported are not limited. Some examples of possible clinical treatments, for which there may be a customized clinical treatment template, could include early-stage hormone-positive breast cancer, metastatic lung cancer, other subtypes or varieties of cancer, autoimmune conditions (e.g., lupus), rheumatoid arthritis, type 1 or type 2 diabetes, cardiovascular diseases, inflammatory bowel disease (IBD), and the like. The method could additionally or alternatively be used for other health conditions or issues. For example, clinical treatment templates could be made for women's health conditions such as endometriosis, PCOS, menopause, pregnancy, infertility treatments, and the like. As another example, a clinical treatment templates could be made for mental health conditions such as depression, PTSD, generalized anxiety disorders, substance abuse, Alzheimer's, and/or other mental health conditions. Other health conditions could similarly be managed using the method. In some variations, the method is used to concurrently manage a variety of different health conditions by the same or multiple treatment teams and other users, but alternatively, the method may be implemented for a single type of clinical treatment.

In some variations, clinical treatment templates may be based in part on the dynamic processing of care guidelines from one or more sources. Accordingly, the method may include retrieving and processing clinical treatment resources to update or generate a clinical treatment template. This may use processing systems using Machine Learning (ML)/ Artificial Intelligent (AI) or other analysis processes to analyze data and care documentation. Extracted information and changes in recommendations may be used to update a clinical treatment template dynamically. For example, as new treatments are added or modified, this processing system may detect the changes in an external resource and then update components of the systems. For example, the guidelines in cancer are updated when new data comes out and medical information changes with high frequency (e.g., every 73 days), the processing system may be used to automatically keep the various clinical treatment templates updated. The updates may reflect changes in guidelines, clinical practice, and U.S. Food and Drug Administration (FDA) drug approvals to ensure the pathway map remains aligned with the latest medical standards and regulatory requirements.

The digital platform for the pathway modeling system includes customization for a patient. The digital platform may additionally include customized features and/or user interfaces for other types of users who may have an interest in the clinical treatment paths of one or more patients. Accordingly, providing the pathway modeling system may additionally include providing an account management system. This may be used to support a variety of different types of accounts such as accounts for patients, doctors, caregivers, family members, third parties (e.g., insurance companies, pharmaceutical companies, counseling services or groups, hospital organizations), and/or other types of accounts.

Different user interface views and modes are customized to the varying types of accounts, which may function to scope permissions, organize data/information, and/or provide other functionality adjustments according to the type of account.

Displaying the pathway map and a history of navigating stages within the pathway map is based at least in part on account type of a user accessing the pathway modeling system. These variations may involve exposing different views and/or different actions.

In one example, a patient view/mode may have a personalized view of their clinical treatment path. A doctor (or any other member of the treatment team, i.e. health professional such as a nurse, case manager, or informal care partner such as a peer navigator, caregiver, etc.) may have a more medically detailed view of a patient's clinical treatment path. For example, a doctor account may additionally have features to perform actions that help guide a patient.

In another example, a doctor's view/mode may also enable features for interacting with a plurality of patients such as switching between different patients and more efficiently seeing and making updates across multiple patients.

In another example, a third-party account for an insurance company or pharmaceutical company may be able to see anonymized views of a patient's treatment path (removal of personally identifiable information) and/or group/population views (e.g., seeing analytics and/or summaries of groups of users).

In some variations, the method may include setting access controls, which functions to enable sharing of a pathway map and/or setting permissions for a pathway map (e.g., view-only, editor access, administrator access). For example, a patient may be able to share access with a family member or a doctor.

In some implementations, there may be a variety of different types of users with different abilities to understand medical information. In some variations, information and data in a clinical treatment template (or in other elements of the digital platform), may be dynamically modified according to a current user. More specifically, source information from a clinical treatment template and other elements of the digital platform may be dynamically adjusted in the technical level of information for different accounts. A doctor or medical staff member may be shown the original technical information, and a user (without any technical or medical background) may have the technical information reformatted to a lower technical information level (e.g., a 7^{th} grade reading level). Additionally, the language used may be dependent on the user's native language, and the platform would dynamically translate the information and pathway maps to the user's preferred language. This may include processing using a large language model, a generative text model, and/or any other ML/AI model or process.

In various examples, the digital platform may incorporate various personalization features to address the diverse needs and preferences of its users, ensuring the accessibility and relevance of medical information across different backgrounds. For instance, content delivery may be customized to meet user preferences for information consumption, such as visual aids, written content, auditory explanations, or interactive video demonstrations. The platform also considers racial, ethnic, and socio-economic backgrounds by providing culturally and socioeconomically relevant resources.

Providing the pathway modeling system may additionally include other aspects related to providing a digital platform. This may additionally or alternatively include providing interfaces with mobile apps, web apps, and/or other types of client devices.

In another variation, this may additionally or alternatively include integrating with external databases such as medical data record systems or other data systems. For example, electronic medical records (EMRs), payor portals, prescription systems, clinical trial systems, electronic clinical decision-making systems, insurance portals, digital health APIs (e.g., Apple or Google health tracking systems), calendars, other healthcare data systems, and/or other data systems may be dynamically accessed and used to update parameters of a patient's state within the pathway map.

In another variation, this may additionally or alternatively include providing or enabling data protection and security systems.

In another variation, this may additionally or alternatively include providing or implementing other components involved in medical-related digital platforms.

Block S120, which includes providing at each stage a set of modules, and functions to enable different resources and user interface options at different stages. A stage is preferably used to provide relevant resources to a user. The stage may also be used to collect input to update parameters. Depending on the implementation, one or more stages of the pathway map may be updated or modified based on various parameters, which may include, without limitation, direct user input (e.g., patient-reported outcomes, physician assessments, lab test results, or other clinical data entries) or external database updates (e.g., treatment guideline updates, health records updates, research findings), and/or the like. Accordingly, providing the set of modules may include providing user resources and/or parameter input interface. These parameters may impact updating the state to a new stage. For example, when a patient decides to proceed with a particular treatment option, that information may be entered and used to move the user's current state to a new stage.

Providing resources may be used to present information, access to multimedia resources like videos, diagrams, and documents. The resources could also include digital interactive resources such as chat interfaces, coaching sessions, digital surveys, and the like. In some variations, this may include providing access to physical products (e.g., wigs at chemotherapy), services (e.g., therapist, or nutritionist), locally available resources (e.g., local non-profit support group), and/or sponsored ads (e.g., sponsored drug ads).

In particular, a stage may provide specific tools to help in managing and understanding the current state of their clinical treatment. For example, a set of questions may be generated for a patient to include in a digital notebook. The patient can use these questions during their medical appointments. In some variations, notes, questions, or patient input can automatically be communicated and synchronized with an EMR system so that a medical professional can easily view information during an appointment.

Providing parameter input interfaces may be used in receiving user input. This input may be used to store notes, update treatment parameters and dates, document test results, and/or update other data related to the clinical treatment. Some of the parameters may be updated through a programmatic interface such as an API to a medical data record system such that a user is not required to update information directly within the pathway modeling system. Receiving input for at least one parameter may result in updating stage (“station”) status. Additionally or alternatively, the digital platform may communicate or synchronize with external systems. For example, this may be used to update an EMR system based on input to the digital platform.

The method may implement a variety of different types of modules, which may be accessed through a stage (or through other suitable channels in the digital platform). Some of those modules may be pre-defined and integrated by default. Other modules may be optional add-ons. Some modules may be customized or designed based in part by external third parties.

As one example, the method may integrate a self-management module that can function as a digital appointment notebook. The self-management module may provide a user interface that enables the patient or family member to prepare for upcoming medical appointments and populate the users' own new or existing digital calendars. The method may dynamically populate suggested questions for upcoming clinical appointments based on where the user is on the pathway map. Personalized questions may also be generated based on historical patterns and other information collected through the digital platform The module enables the user to take notes in the appointment and identify follow-up care.

As another example, the platform may offer family-management modules for non-clinical areas of support such as nutrition. In this example, the module may offer templates to enable the caregiver and patient to collaborate and identify which nutrition items can or cannot be consumed. The platform then dynamically offers recipes and grocery lists based on the users input.

Block S130, which includes updating the stage of a patient within the pathway modeling system according to parameters of treatment decisions made within at least one stage, functions to dynamically update the pathway map to reflect the current state of a patient's treatment. Updating stage of a patient may include updating to a current state to a new stage. The new stage may generally be a stage following the current stage. The new stage however may be a previous stage, or it may jump to a different stage if the treatment follows a non-linear path.

Updating the stage of a patient is preferably performed repeatedly over the course of a treatment. Moving to a new stage will preferably trigger or lead to the presentation of new resources and requests for new parameter inputs. As treatments are not always linear, a patient may move through stages or paths of the pathway map repeatedly or even in a recursive manner. For example, in metastatic breast cancer, the pathway may be recursive until death.

Processes of the method (e.g., Block S110, S120, and S130) are preferably iteratively performed such that the clinical treatment navigation system is updated to reflect the current status and decisions.

In some variations, the method may be modified to support one patient managing multiple pathway maps if, for example, they have multiple clinical treatments occurring at the same time. In one variation, the maps would be managed as distinct maps. In another variation, pathway maps may be merged such that they can view in single interface information from two treatments in a unified user interface.

In some variations, the method may include configuring or providing a customized module, which functions to enable capabilities to customize the user interface of the pathway modeling system to suit the individual desires of a user. Examples of customized modules may include an insurance module, a pharmaceutical module, a social/communication module, and other self-management and family-management modules. A module can preferably be customized and offered through the digital platform. A user may be enabled to turn on and off different modules.

As an example of customizing for insurance coverage, an insurance module may be configured to automatically reflect insurance-related details within each stage. This may be used to show coverage levels for different treatment options. This may enable patients and doctors to weigh the financial impact of different options.

As an example of customizing for pharmaceutical-related features, a pharmaceutical module may add resources related to one or more different pharmaceutical options. This may be used to help in identifying treatment opportunities, providing better contextual guidance in the use of existing treatment options (instructions on administrating pharmaceuticals), offering data relevant to a patient for a possible drug option based on their treatment journey (e.g., information about specific treatment options or connecting to resources provided through a pharmaceutical company or its partners), side effects of treatment selection, and/or other information.

As another example of customizing with more digital/social features, a social connection module may be enabled that provides dynamic connections and communication tools. These may enable patients to dynamically connect with others with shared treatment experiences. This can be automatically facilitated without requiring patients to share sensitive information.

### 3. System

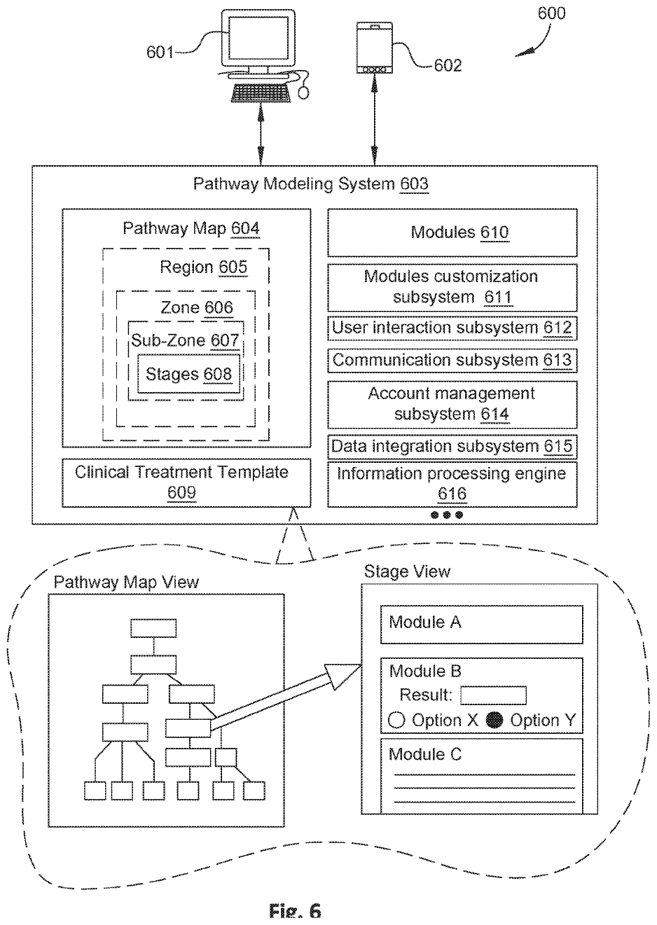

FIG. 6 is a schematic representation of system 600 for navigating and managing clinical care continuum according to embodiments of the subject technology. This diagram is merely an example, which should not unduly limit the scope of the claims. One of ordinary skill in the art would recognize many variations, alternatives, and modifications. In various implementations, system 600 may be implemented by a computing system comprising one or more computing devices (e.g., computer 601 or smartphone 602). System 600 may be operationalized through various forms of hardware and software implementations, such as computer application programs, application programming interfaces (APIs), libraries, and/or other computer-program product formats designed to carry out the tasks associated with managing clinical care continuum.

As shown in FIG. 6, system 600 for a digital platform assisting in managing clinical care continuum may include a pathway modeling system 603 that includes a model of a pathway map 604 with a set of clinical stages 608 based on a clinical treatment template 609; and a set of modules 610 accessible through a user interface at each stage. The system may additionally include module customization subsystem 611 to customize and/or modify the modules of one or more stages 608.

The pathway modeling system 603 functions as a digital platform, managing all aspects of the system's user interface and enabling users to access the platform from a wide range of client devices, such as web browsers, mobile apps, or other computing devices. In some embodiment, system 600 may include user interaction subsystem 612 configured to display pathway map 604 and/or receive user inputs. For instance, user interaction subsystem 612 includes a display device and/or a user input device.

In some embodiments, system 600 may include an account management subsystem 614 to enable users to create and manage their account information. Account management subsystem 614 may be configured to manage user profiles and permissions for accessing pathway map 604. System 600 may additionally or alternatively include data integration subsystem 615 configured to integrate with the external database and/or perform other forms of data integration such as with electronic medical records (EMRs), payor portals, prescription systems, insurance portals, digital health APIs (e.g., Apple or Google health tracking systems), calendars, other healthcare data systems, and/or other data systems that may either serve as data inputs or outputs of the system.

The pathway modeling system 603 preferably manages operating data systems, application state, and user interfaces used in providing a navigational map of the treatment journey represented through the pathway map, stage components, and various stage modules.

The pathway map 604 functions in defining a mapping between stages of a treatment journey. The pathway map 604 can be based on a clinical treatment template 609. The pathway map 604 models paths between different clinical stages and is designed to provide a detailed visual representation of options. As a visual-spatial map, pathway map 604 can give a patient or other user a better understanding of where they are in the clinical process with insight into what has currently happened, what are important details to deal with now, and what is upcoming. In some implementations, the pathway map 604 can be zoomed in or out to provide more or less detail to the user depending on their specific needs at that moment.

In various implementations, pathway map 604 may be designed with a hierarchical structure including region 605, zone 606, and/or sub-zone 607. The regions, zones, or other types of sub-regions and sub-zones may function as groupings of related stages. The hierarchical design of pathway map 604 not only simplifies the navigation of complex medical information but also enhances patient understanding and engagement with their clinical process. By breaking down the complex medical journey into categorized stages, patients can easily identify their current status, upcoming steps, and the resources available at each point. The user interface may update to reflect a patient moving through these different regions. In some examples, sub-zone 607 may include one or more stages 608 to provide targeted information, resources, and tools for treatment planning and management.

The stage components 608 function as representations of different points along pathway map 604, representing key states in the patient's treatment journey. Each stage may present resources that may be relevant to the user at that particular point in time and offer user interfaces to confirm treatment decisions or collect other relevant data. These resources may be provided or defined in part through one or more modules. In some implementations, stages may be grouped together into larger sections or pathways, with each pathway representing a broader phase of the treatment journey. The stages may be customized based on the user's specific healthcare condition, helping to ensure that the system is tailored to the patient's individual needs and requirements. Overall, the stages 608 serve as the primary means by which users interact with the platform, allowing them to progress through their treatment journey with confidence and ease. In various examples, stage 608 may include a self-management module or a family-management module including, without limitation, an insurance module, a pharmaceutical module, a social/communication module, and/or the like.

The clinical treatment template 609 serves as a configuration file(s) that defines the data and options for the pathway map. System 600 may have one or more clinical treatment templates, each of which may correspond to a specific type of clinical treatment. For example, there may be different templates for various cancer treatments, inflammatory bowel disease, pregnancy, and other long and complex treatment journeys.

In some variations, system 600 includes a clinical treatment configuration component that allows administrators to create, edit, or modify clinical treatment templates in a convenient interface. This customization can help ensure that the pathway map is tailored to the needs of each individual patient.

In some variations, clinical treatment templates 609 may be based in part on dynamic processing of care guidelines from one or more sources. Processing systems using Machine Learning (ML)/Artificial Intelligent (AI) or other analysis processes may be used to analyze data and care documentation and dynamically update a clinical treatment configuration. For example, as new treatments are added or modified, this processing system may detect the changes in an external resource and then update components of the systems. For example, the guidelines in cancer are updated when new data comes out and medical information changes with high frequency (e.g., every 73 days), the processing system may be used to automatically keep the various clinical treatment templates updated.

In some implementations, system 600 may include an information processing engine 616. Information processing engine 616 may be used to dynamically modify the language used to communicate concepts. In some examples, information processing engine 616 may be configured to perform content adaptation within pathway map 604 based on user preference. This may be used to simplify technical information for users without a technical background. The information processing engine 616 may include or interface with a large language model, a generative text model, and/or any other ML/AI model or process. The processing engine 616 can take in source information and output a summarization, simplified description, or provide supplemental explanation.

The modules 610 function to define the resources or interfaces provided at each stage of the pathway map 604. The modules 610 can be customized based on the specific healthcare condition being treated, helping to ensure that patients receive the support and guidance they need throughout their treatment journey.

In some implementations, system 600 may include a module customization subsystem 611, which provides a user interface and data system that enables one or more customizations to the platform. For instance, module customization subsystem 611 may be configured to update stage 608 based on user input. For example, the module customization subsystem 611 could enable insurance providers to dynamically reflect insurance coverage for different treatment options and modify other aspects of the platform that may be relevant to a patient's insurance provider. Ultimately, modules 610 serve as a key mechanism for providing personalized support to patients, helping them navigate their treatment journey with greater ease and confidence.

### 4. System Architecture

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

In one variation, a system comprising of one or more computer-readable mediums (e.g., non-transitory computer-readable mediums) storing instructions that, when executed by the one or more computer processors, cause a computing platform to perform operations comprising those of the system or method described herein such as: providing a pathway modeling system that models possible paths through stages of a clinical treatment; providing at each stage a set of modules; and updating stage of a patient within the pathway modeling system according to parameters of treatment decisions made in at least one stage.

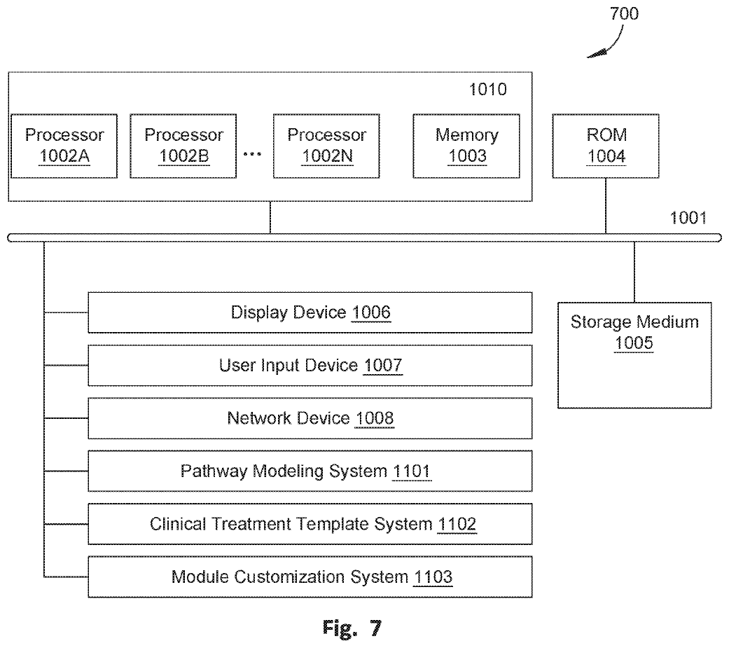

FIG. 7 is an exemplary system architecture 700 that may be used in implementing the system and/or method according to embodiments of the subject technology. In some implementations, system 700 is implemented in a plurality of devices in communication over a communication channel and/or network. In some implementations, the elements of the system are implemented in separate computing devices. In some implementations, two or more of the system elements are implemented in same devices. The system and portions of the system may be integrated into a computing device or system that can serve as or within the system.

The communication channel 1001 interfaces with the processors 1002A-1002N, the memory (e.g., a random access memory (RAM)) 1003, a read-only memory (ROM) 1004, a processor-readable storage medium 1005, a display device 1006, a user input device 1007, and a network device 1008. As shown, the computer infrastructure may be used in connecting pathway modeling system 1101, clinical treatment template system 1102, module customization system 1103, and/or other suitable computing devices.

In some implementations, storage medium 1005 may be configured to store a first database. For example, the first database may include a collection of first data, each set of data being associated with specific clinical information, such as those for different types of cancers or other diseases. The structured organization within the first database enables processing unit 1010 to efficiently retrieve and synthesize pertinent information, which is then utilized by pathway modeling system 1101 to generate a visual representation of a patient's treatment journey. This visual representation, or pathway map, not only reflects the hierarchy of clinical stages but is also dynamically tailored to individual patient needs based on the specific guidelines related to their medical condition.

In some embodiments, the accessibility and structure of the first data within the first database may streamline the process of updating the pathway map. As new guidelines are introduced or existing ones are revised, the first database can be promptly updated, which in turn allows pathway modeling system 1101 to reflect the most current medical protocols. This helps to ensure that healthcare providers and patients are always accessing the latest recommendations, enhancing the decision-making process and potentially improving patient outcomes. In some examples, storage medium 1005 may be structured to facilitate rapid retrieval and efficient data management. This allows pathway modeling system 1101 to access and integrate the necessary information seamlessly, minimizing latency and enhancing the system's overall responsiveness.

The processors 1002A-1002N may take many forms, such CPUs (Central Processing Units), GPUs (Graphical Processing Units), microprocessors, ML/DL (Machine Learning/Deep Learning) processing units such as a Tensor Processing Unit, FPGA (Field Programmable Gate Arrays, custom processors, and/or any suitable type of processor.

The processors 1002A-1002N and the main memory 1003 (or some sub-combination) can form a processing unit 1010. Processing unit 1010 may be coupled to storage medium 1005 and configured to retrieve the first data from storage medium 1005. Processing unit 1010 may generate a pathway map using at least the first data. For example, the pathway map represents a hierarchy of clinical stages within a first clinical process. This mapping translates complicated medical protocols and treatment guidelines into a structured, interactive format that enhances user comprehension and facilitates clinical decision-making.

In various implementations, display device 1006 may be configured to display the pathway map. It allows users to interact with the visual components of system 700, presenting a user-friendly interface for complex data such as pathway maps and treatment options.

In some embodiments, the processing unit 1010 includes one or more processors communicatively coupled to one or more of a RAM, ROM, and machine-readable storage medium; the one or more processors of the processing unit receive instructions stored by the one or more of a RAM, ROM, and machine-readable storage medium via a bus; and the one or more processors execute the received instructions. In some embodiments, the processing unit is an ASIC (Application-Specific Integrated Circuit). In some embodiments, the processing unit is a SoC (System-on-Chip). In some embodiments, the processing unit includes one or more of the elements of the system.

In various implementations, user input device 1007 may be configured to receive a first user input, which may include, without limitation, stage identification, medical diagnosis, diagnostic test result, treatment decision, symptom report, user preference, and/or the like. User input device 1007 may include, without limitation, a keyboard, touchscreen, mouse, voice recognition, and/or other sensor-based inputs. By accommodating diverse input methods, system 700 expands its utility across different user contexts and preferences, ensuring accessibility for users with varying levels of technological proficiency.

Depending on the implementation, processing unit 1010 may update or modify the pathway map based on various parameters such as direct user input or external database updates. In an example, processing unit 1010 may be configured to update the pathway map based on a first parameter associated with the first user input. In other examples, processing unit 1010 may be configured to update the pathway map based on a second parameter associated with an update of the first data (e.g., treatment guideline updates, health records updates, research findings, and/or the like).

A network device 1008 may provide one or more wired or wireless interfaces for exchanging data and commands between the system and/or other devices, such as devices of external systems. Such wired and wireless interfaces include, for example, a universal serial bus (USB) interface, Bluetooth interface, Wi-Fi interface, Ethernet interface, near field communication (NFC) interface, and the like. Network device 1008 may support high-speed data transfer and secure communication protocols, ensuring that sensitive medical information is exchanged with integrity and confidentiality across the healthcare network.

In an example, network device 1008 may be configured to communicate with a second database, which may include a second data associated with patient-specific information. The patient-specific information comprises at least one of a medical history, genetic information, biomarker information, diagnostic results, current medications, allergy information, financial information, insurance information, and/or the like. For instance, biomarker information may include, without limitation, human germ line genetic and genomic information, tumor or blood genetic and genomic information, blood-based tumor markers, circulating tumor DNA, RNA and its modifications, formed elements of the peripheral blood, blood or plasma proteins and protein subunits to the amino acid level, and blood chemistry, immunohistochemistry determinations from the tumor and its components including malignant cells and the surrounding stroma, and/or other indications of genotype and phenotype. The system's ability to communicate with external databases via network device 1008 ensures that patient-specific information is continually updated and synchronized with the pathway map. For example, when a patient's electronic health record is updated with a new lab result or medication change, network device 1008 ensures that such update is promptly reflected in the pathway map, contributing to an up-to-date treatment management system.

Computer and/or Machine-readable executable instructions comprising of configuration for software programs (such as an operating system, application programs, and device drivers) can be stored in the memory 1003 from the processor-readable storage medium 1005, the ROM 1004 or any other data storage system. For example, memory 1003 may be configured to store the pathway map and/or other related data structures, enabling quick data retrieval, updates, and manipulations by processing unit 1010.

When executed by one or more computer processors, the respective machine-executable instructions may be accessed by at least one of processors 1002A-1002N (of a processing unit 1010) via the communication channel 1001, and then executed by at least one of processors 1001A-1001N. Data, databases, data records or other stored forms data created or used by the software programs can also be stored in memory 1003, and such data is accessed by at least one of processors 1002A-1002N during the execution of the machine-executable instructions of the software programs.

The processor-readable storage medium 1005 is one of (or a combination of two or more of) a hard drive, a flash drive, a DVD, a CD, an optical disk, a floppy disk, a flash storage, a solid-state drive, a ROM, an EEPROM, an electronic circuit, a semiconductor memory device, and the like. The processor-readable storage medium 1005 can include an operating system, software programs, device drivers, and/or other suitable sub-systems or software.

The combination of hardware and software components of system 700 not only streamlines the treatment navigation process but also introduces a level of efficiency and personalization in clinical treatment management. By reducing the cognitive load on healthcare providers and patients, system 700 improves the functionality and responsiveness of the computing environment in which it operates, addressing a critical need in the field of digital health solutions.

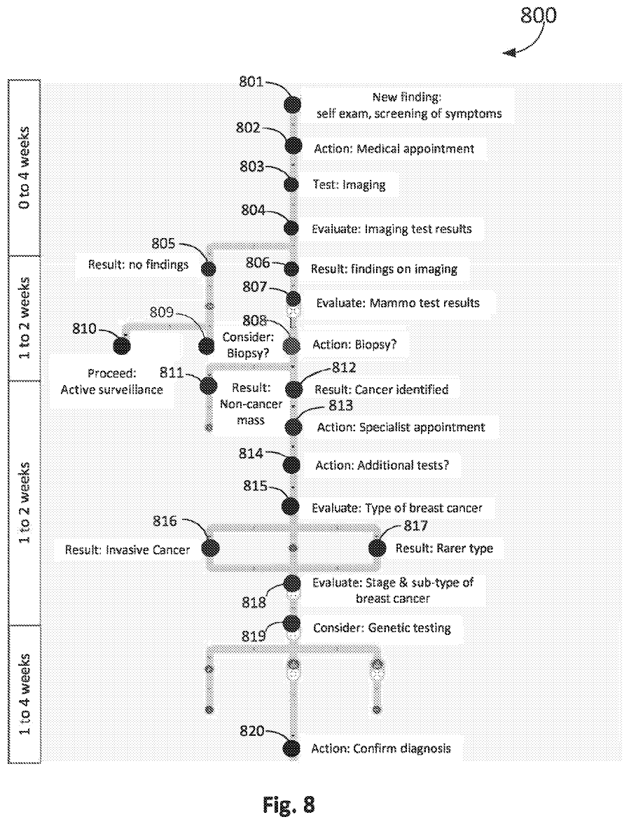

FIG. 8 is an exemplary pathway map segment 800 illustrating a diagnosis phase for early-stage estrogen receptor (ER)/progesterone receptor (PR) positive (ER+/PR+) breast cancer according to embodiments of the subject technology. This diagram is merely an example, which should not unduly limit the scope of the claims. One of ordinary skill in the art would recognize many variations, alternatives, and modifications. This map segment provides a time-based visualization of the sequential steps and decision points from initial findings to diagnosis confirmation during early-stage ER/PR breast cancer diagnosis. For instance, the vertical axis of the map provides timeline information, establishing a chronological flow that guides the user through a sequence of healthcare interactions and clinical assessments in the early detection and diagnosis of breast cancer. In various applications, each point or marker on the pathway map may include estimates of time spent within each station, reflecting the duration of treatments associated with each phase of care. These estimates may be presented as static durations (e.g., 6 weeks for recovery from surgery) or as dynamic durations (e.g., where the user inputs the number of three-week treatment cycles, and an estimated duration is computed). The duration of each station within a subzone may be summed, either statically or dynamically. Based on user input, dates may be attached to each time point and duration, and used to project the timing of future events.

In various examples, pathway map 800 begins at stage 801 with the patient's identification of a new finding during a self-exam or screening of symptoms, prompting an action at stage 802, where a medical appointment is scheduled. Following the appointment, stage 803 indicates an imaging test is conducted. The results of the imaging test are then evaluated at stage 804. For instance, pathway map 800 may present two potential outcomes: no findings at stage 805 or new findings at stage 806.

At each stage, the user may be provided with relevant information such as potential implications of the findings, recommended follow-up procedures, and preparatory guidelines for the next steps. If no findings are present, as indicated at stage 805, the pathway continues to stage 808, which involves considering a biopsy. Alternatively, the user may choose active surveillance as a prudent course of action at stage 810. If findings on the imaging are noted at stage 806, the pathway advances to stage 807 where the imaging test results are further evaluated (e.g., via another breast imaging technique).

To enhance the user experience, pathway map 800 may incorporate a dynamic positioning feature that visually pins the user's current stage within the map and progressively reveals potential treatment paths based on user selections and preferences. This dynamic visual guidance provides a more personalized navigation experience by adapting the displayed pathway to reflect the user's progress and choices, thereby enhancing the overall navigability and utility of the pathway map.

According to some embodiments, pathway Map 800 may incorporate a search feature that allows users to input one or more search terms, such as “radiation.” Upon receiving a search term, the system may highlight all relevant stations on the pathway map that correspond to the queried term, thereby facilitating quick access to pertinent information. As the user interacts with the pathway map, the system may intelligently respond to the search terms or questions provided by the user. For example, if a user searches for “radiation,” the system may highlight the pathways and stations related to radiation treatment, facilitating easy access to relevant information.

In the event that the mammogram results raise concerns, as determined by the assessment at stage 807, the pathway leads to stage 808, which presents the user with the option of proceeding with a biopsy to gather further diagnostic information. A negative biopsy result at stage 811 indicates a non-cancerous mass, which may lead to further evaluation and monitoring. Conversely, if a cancer is identified at stage 812, the pathway then directs the user to specialist consultation at stage 813. Based on the specific diagnosis, stage 813 may offer recommendations or refer patients to specialists for a more comprehensive evaluation. The specialist consultation may result in additional tests being recommended at stage 814 to further understand the type and nature of the cancer. This is followed by stage 815, where the type of breast cancer is evaluated and determined. For instance, stage 816 indicates the identification of invasive cancer, and stage 817 denotes the identification of a rarer type of cancer. The stage and subtype of breast cancer are further assessed at stage 818. Based on these assessments, genetic testing might be considered at stage 819 as a means to further personalize treatment strategies.

Finally, the diagnosis phase concludes at stage 820, where all information is synthesized to confirm the diagnosis. Depending on the diagnosis, the pathway may direct the user to other regions of the map for subsequent stages of treatment planning and management. For instance, the subsequent areas may include treatment zones focused on the specifics of therapeutic approaches, where patients can explore options such as surgery, chemotherapy, radiation, and/or the like. Pathway map 800 provides a tailored navigation tool that ensures that patients are guided through their unique treatment landscapes with information that is relevant to their diagnosed condition and personal preference.

As used herein, first, second, third, etc. are used to characterize and distinguish various elements, components, regions, layers, and/or sections. These elements, components, regions, layers, and/or sections should not be limited by these terms. Use of numerical terms may be used to distinguish one element, component, region, layer, and/or section from another element, component, region, layer and/or section. Use of such numerical terms does not imply a sequence or order unless clearly indicated by the context. Such numerical references may be used interchangeably without departing from the teaching of the embodiments and variations herein.

As a person skilled in the art will recognize from the previous detailed description and from the figures and claims, modifications and changes can be made to the embodiments of the invention without departing from the scope of this invention as defined in the following claims.

We claim:

1. An apparatus for navigating and managing clinical care continuum, comprising:
a storage medium configured to store a first database, the first database comprising a first data associated with first clinical information;
a processing unit coupled to the storage medium and configured to:
retrieve the first data from the storage medium; and
generate a pathway map using at least the first data, the pathway map representing a hierarchy of clinical stages within a first clinical process;
a memory coupled to the processing unit and configured to store the pathway map;
a user interface coupled to the processing unit and configured to receive a first user input; and
a network device coupled to the processing unit and configured to communicate with a second database.

2. The apparatus of claim 1, wherein the pathway map comprises a first stage of the first clinical process, and the first stage comprises a first module configured to provide medical information associated with the first stage and a medical condition of a first patient.

3. The apparatus of claim 2, wherein the medical information comprises at least one of treatment options, educational resources, risk assessments, nutritional guidance, support services, or medication information.

4. The apparatus of claim 1, wherein the processing unit is further configured to update the pathway map based on a first parameter, the first parameter being associated with the first user input.

5. The apparatus of claim 1, wherein the first user input comprises at least one of stage identification, medical diagnosis, diagnostic test result, treatment decision, symptom report, or user preference.

6. The apparatus of claim 1, wherein the processing unit is further configured to update the pathway map based on a second parameter associated with an update of the first data.

7. The apparatus of claim 1, wherein the second database comprises a second data associated with patient-specific information, the patient-specific information comprises at least one of medical history, genetic information, biomarker information, diagnostic results, current medications, allergy information, financial information, or insurance information.

8. The apparatus of claim 1, wherein the user interface further comprises a display device configured to display the pathway map.

9. A system for navigating and managing clinical care continuum, comprising:
a pathway modeling subsystem configured to generate a pathway map representing a hierarchy of clinical stages within a first clinical process, the pathway map comprising a first stage indicative of a first step of the first clinical process;
a user interaction subsystem configured to display the pathway map and receive a first user input;
a module customization subsystem configured to update the first stage based on the first user input; and
a communication subsystem configured to communicate with an external database.

10. The system of claim 9, wherein the first stage comprises a decision station or a scenario station.

11. The system of claim 9, wherein the first stage comprises a first module configured to provide medical information associated with the first stage.

12. The system of claim 11, wherein the medical information comprises at least one of treatment options, educational resources, risk assessments, nutritional guidance, support services, or medication information.

13. The system of claim 9, further comprising an account management subsystem configured to manage user profiles and permissions for accessing the pathway map.

14. The system of claim 9, further comprising a data integration subsystem configured to integrate with the external database, the external database comprises at least one of electronic health records, payor portals, or prescription systems.

15. The system of claim 9, wherein the pathway map is generated based on first clinical information.

16. The system of claim 9, further comprising an information processing engine configured to perform content adaptation within the pathway map based on user preference.

17. A method for navigating and managing clinical care continuum, comprising:
generating, by a processing unit, a pathway map representing a hierarchy of clinical stages within a first clinical process, the pathway map comprising a first stage indicative of a first step of the first clinical process;
displaying, by a user interface, the pathway map;
receiving, by the user interface, a first user input; and
updating, by the processing unit, the pathway map based on a first parameter associated with the first user input.

18. The method of claim 17, further comprising:
providing, by the user interface, medical information associated with the first stage.

19. The method of claim 18, wherein the medical information comprises at least one of treatment options, educational resources, risk assessments, nutritional guidance, support services, or medication information.

20. The method of claim 17, wherein the first user input comprises at least one of stage identification, medical diagnosis, diagnostic test result, treatment decision, symptom report, or user preference.
